(19) United States
(12) Patent Application Publication
(10) Pub. No.: US 2010/0324827 A1
(43) Pub. Date: Dec. 23, 2010

(54) Fluid Responsiveness Measure

(75) Inventors: Paul Stanley Addison, Edinburgh (GB); James Nicholas Watson, Dunfermline (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue (IE)

(21) Appl. No.: 12/486,915

(22) Filed: Jun. 18, 2009

(51) Int. Cl. A61B 5/0452; A61B 5/02; A61B 5/0488; G06F 19/00

(52) U.S. Cl. 702/19; 600/561; 600/509; 600/546; 600/515

(57) ABSTRACT

A method and system for measuring fluid responsiveness of a patient is disclosed. Information related to fluid responsiveness of a patient may be derived from a PPG signal, for example, by analyzing the PPG signal transformed by a continuous wavelet transform. Other techniques for deriving information related to fluid responsiveness of a patient include, for example, analyzing the amplitude modulation, frequency modulation, and/or baseline changes of a PPG signal.

The present disclosure relates to physiological signal processing and, more particularly, the present disclosure relates to processing physiological signals to determine the fluid responsiveness of a patient.

### SUMMARY OF THE INVENTION

Respiratory variation of the photoplethysmograph signal may correlate with fluid responsiveness. For example, manually measuring the height of the pulse component of the photoplethysmograph signal by eye may provide a clinician with information regarding the fluid responsiveness of a patient.

In one suitable approach, wavelet transforms may be used to better determine characteristic metrics of the respiratory components in the photoplethysmograph signal, which may then be correlated with fluid responsiveness. For example, respiratory components may be extracted from the photoplethysmograph signal using wavelet analysis. The amplitude modulation of the photoplethysmograph signal may be taken directly from the amplitude modulation of the pulse band ridge, which manifests itself in the wavelet transform. By measuring the amplitude variation of the pulse band ridge, the local pulse modulation may be extracted, and the variation of the local pulse modulation may then be used to indicate the level of fluid responsiveness of a patient. Other components of the photoplethysmograph signal, such as, for example, the dichrotic notch and other waveform reflections, are also present in the wavelet transform space. Changes in the modulation amplitude of these features may also be indicative of changes in fluid responsiveness.

The variation of the respiratory components may be captured by the ratio A/M, where A is the peak-to-peak amplitude of the modulation and M is the mean value of the signal. This measure is called the relative pulse amplitude modulation (RPAM). The RPAM may be used to indicate or taken as a measure of the fluid responsiveness of the patient. Other ratios may be used to define the RPAM, including the use of the standard deviation of the amplitude modulations from the mean, the median absolute value of amplitude modulation from the mean, any other suitable metric that expresses the amplitude modulation to define the RPAM, or any combination thereof. In addition, instead of, or in addition to, using the mean, other characteristic baseline signals may be used, including the lower bound of the signal interpolated from the troughs, the upper bound of the signal interpolated from the peaks, any other suitable characteristic baseline signal, or any combination thereof.

The carrier wave amplitude may also be used to determine the degree of fluid responsiveness of the patient. The carrier wave is indicative of venous return. The carrier wave amplitude may be given by the amplitude of the breathing band in wavelet space. However, because the respiration modulation component as expressed in the breathing band corresponds to the respiration modulation amplitude, this may be divided by a mean signal baseline to provide a ratio from the breathing band components. Thus, the respiratory breathing amplitude modulation (RBAM) may correspond to a property scaled ratio of the breathing band component and the baseline of the signal. For example, this may be performed by computing the inverse wavelet transform of the breathing band components and the inverse wavelet transform of the components below the baseline and dividing the former by the latter.

The respiratory sinus arrhythmia (RSA) component variation may also be used to determine the degree of fluid responsiveness of the patient. RSA is a naturally occurring variation in the periodicity of the heart beat timing over the respiration cycle. The RSA component may be derived from the pulse band ridge in wavelet space. In a method, the modulation of the characteristic scale of the pulse component may be divided by the mean characteristic scale of the pulse to provide a characteristic pulse scale modulation (CPSM). Any band in the transform space indicative of pulse period may provide information for measuring RSA, such as a band at a scale above that of the pulse band, which, though of lower amplitude, may clearly indicate RSA.

These techniques for determining fluid responsiveness may be used in connection with any one or more biosignals including the photoplethysmograph signal: for example the carrier wave, amplitude modulation, and RSA information may be derived from an arterial line trace and used in the determination of fluid responsiveness.

The combination of one or more of the above parameters offers a significant enhancement to current practices in the analysis of fluid responsiveness.

In other examples, part of, or all of, the methodology described above may be used to extract information pertaining to fluid responsiveness of a patient from other biosignals including, for example, the electrocardiogram, electroencephalogram, electrogastrogram, electromyogram, any other suitable biosignal, or any combination thereof.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with an embodiment;

FIG. 4 is a block diagram of an illustrative continuous wavelet processing system in accordance with an embodiment;

FIG. 5 is a flow chart of illustrative steps involved in determining information related to fluid responsiveness in accordance with an embodiment;

FIG. 6 shows an illustrative amplitude modulation waveform in accordance with an embodiment;

FIG. 7 shows an illustrative pulse band ridge extracted from a scalogram of a wavelet-transformed photoplethysmograph signal in accordance with an embodiment;

FIG. 8 shows an illustrative carrier wave in accordance with an embodiment;

FIG. 9(a) shows an illustrative signal exhibiting respiratory sinus arrhythmia in accordance with an embodiment;

FIG. 9(b) shows an illustrative respiratory sinus arrhythmia waveform extracted from the pulse band ridge of a wavelet transform of the signal in FIG. 9(a) in accordance with an embodiment;

FIG. 10 shows an illustrative output device displaying information related to fluid responsiveness in accordance with an embodiment; and

FIG. 11 is a flow chart of illustrative steps involved in determining information related to fluid responsiveness in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambeit-Beer's law, The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the“ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) \text{/} d t}{d log I ( {λ}_{IR} ) \text{/} d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , t ) ,$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5).
Using the relationship

$$\frac{d log I}{d t} = \frac{d I \text{/} d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR}) (8)

y(t)=Rx(t)

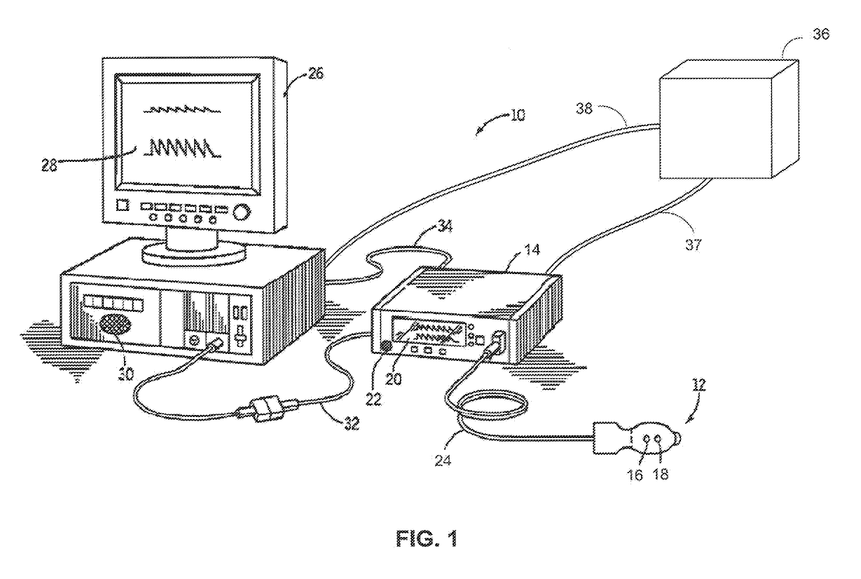

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patients forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

In the illustrated embodiment, pulse oximetry system 10 may also include fluid delivery device 36, which delivers fluid to patient. Fluid delivery device 36 may be an intravenous line, an infusion pump, any other suitable fluid delivery device, or any combination thereof that delivers fluid to a patient. The fluid delivered to a patient may be saline, plasma, blood, water, any other fluid suitable for delivery to a patient, or any combination thereof. Fluid delivery device 36 may be configured to adjust the quantity or concentration of fluid delivered to a patient.

Fluid delivery device 36 may be communicatively coupled to pulse oximetry monitor 14 via a cable 37 that is coupled to a digital communications port or may communicate wirelessly (not shown). Alternatively or in addition, fluid delivery device 36 may be communicatively coupled to multi-parameter patient monitor 26 via a cable 38 that is coupled to a digital communications port or may communicate wirelessly (not shown).

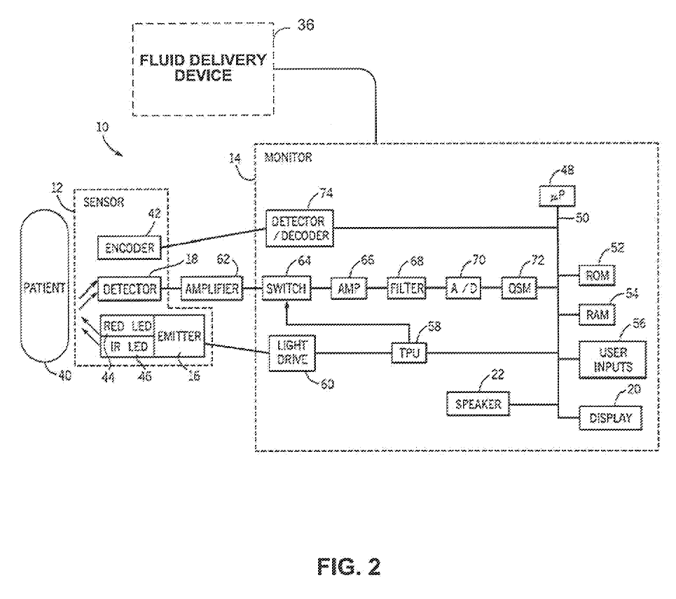

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patients tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patients tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patients tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

In an embodiment, fluid delivery device 36 may be communicatively coupled to the monitor 14. Microprocessor 48 may determine the patient's physiological parameters, such as a change or level of fluid responsiveness, and display the parameters on display 20. In an embodiment, the parameters determined by microprocessor 48 may be used to adjust the fluid delivered to the patient via fluid delivery device 36.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be resealed for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear resealing, any power of the modulus of the wavelet transform, or any other suitable rescaling. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c} the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{12πf}^{₀}^{t}−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 {πf}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

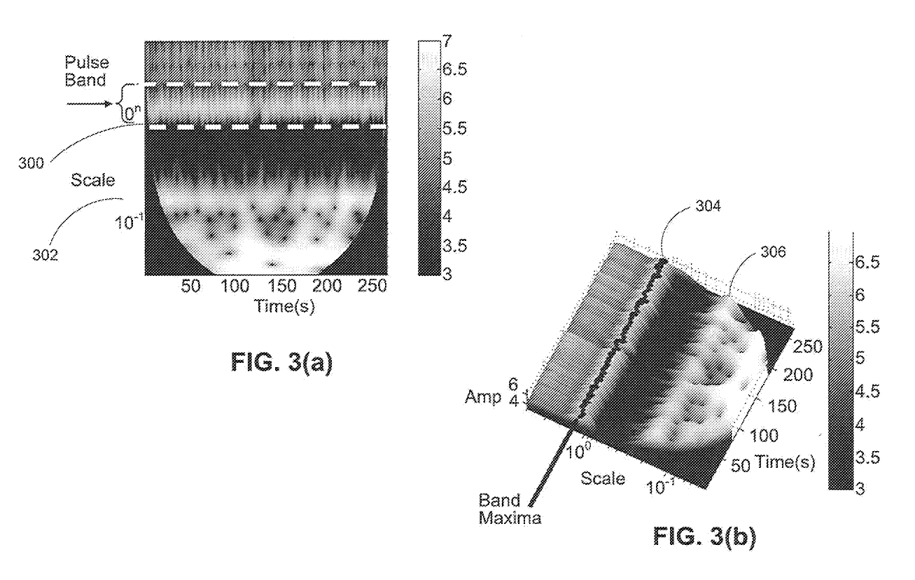

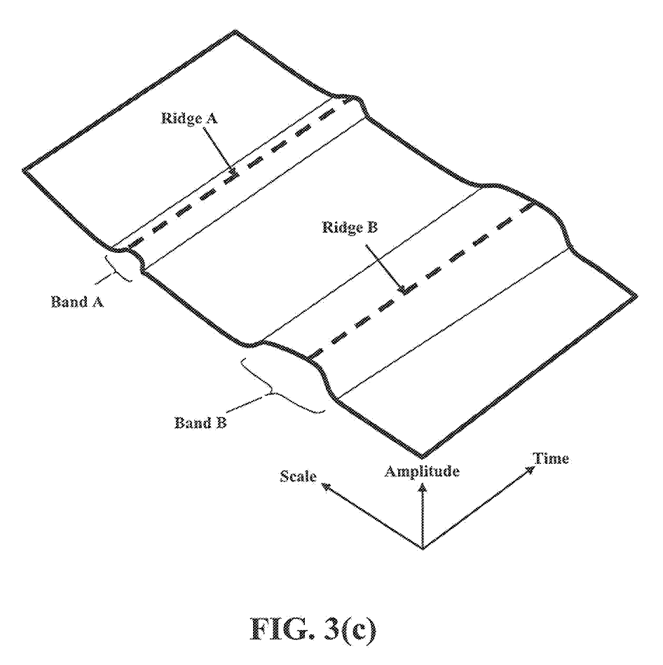

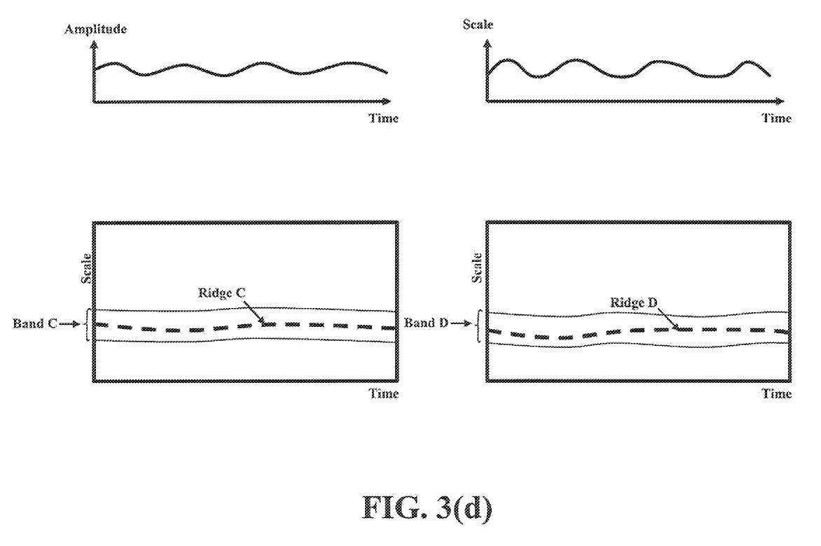

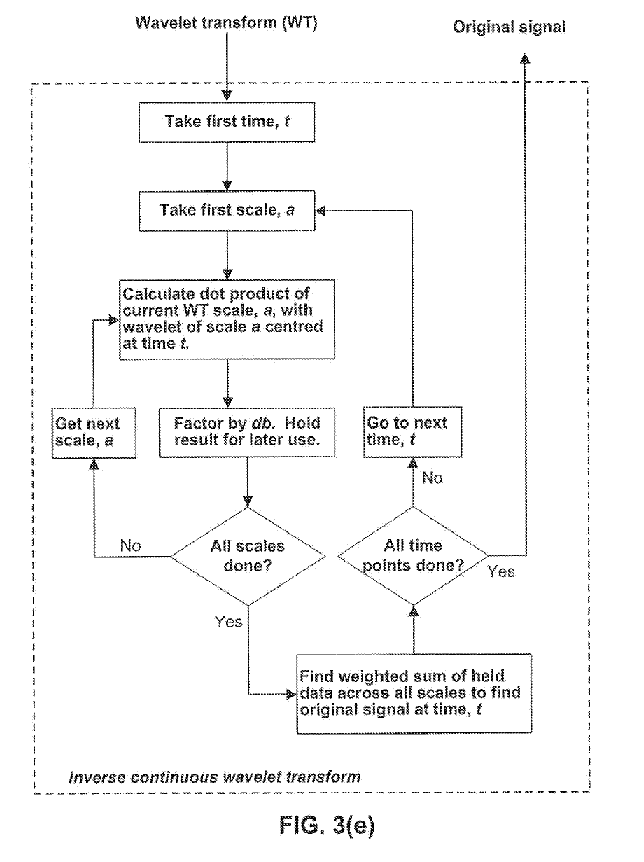

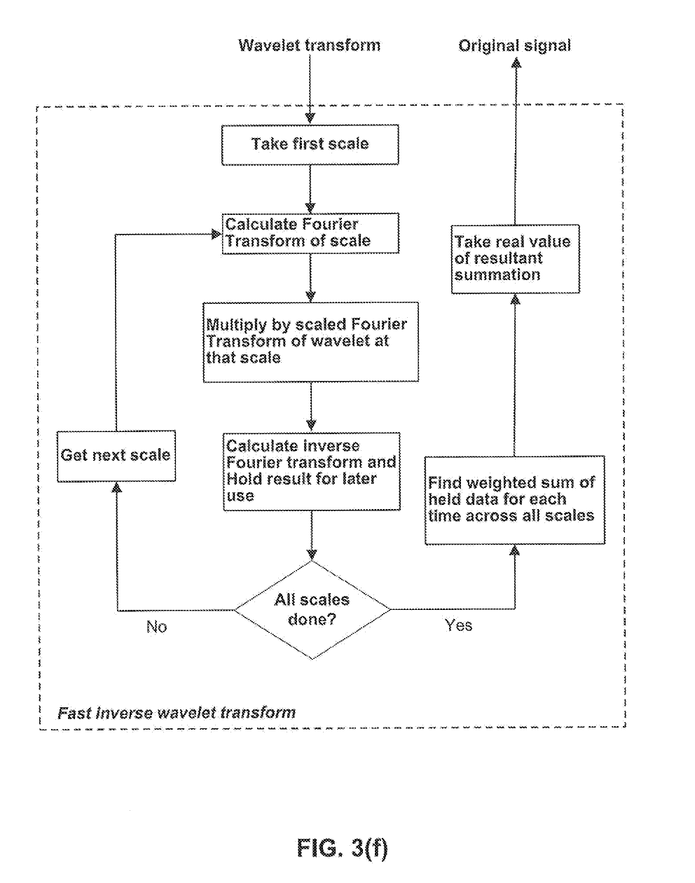

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a rescaled wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

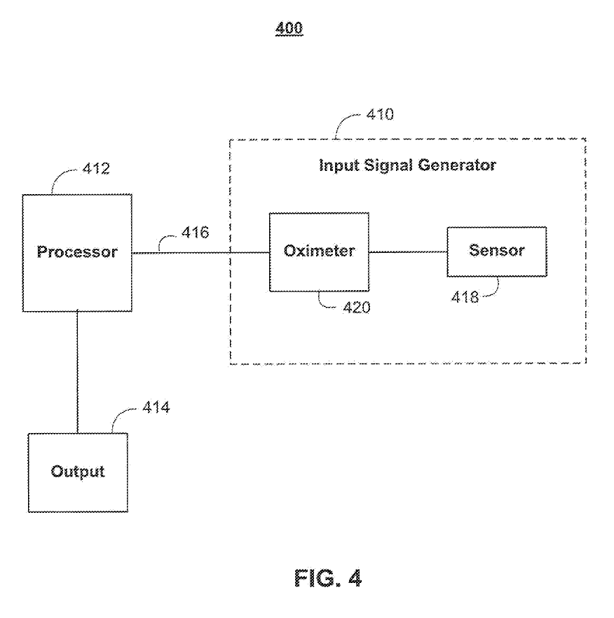

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

In an embodiment, the present disclosure may be used in determining the fluid responsiveness of a patient. Fluid responsiveness may be monitored in, for example, critically ill patients because fluid administration plays an important role in optimizing stroke volume, cardiac output, and oxygen delivery to organs and tissues. However, clinicians must often balance between central blood volume depletion and volume overloading. Critically ill patients are at greater risk for volume depletion and severe hypotension is a common life-threatening condition in critically ill patients. Conversely, administering too much fluid can induce life-threatening adverse effects, such as volume overload, systemic and pulmonary edema, and increased tissue hypoxia. Therefore, obtaining reliable information and parameters that aid clinicians in fluid management decisions may help improve patient outcomes.

Respiratory variation of the PPG signal may correlate to the fluid responsiveness of a patient. Respiratory variation may be determined by, for example, manually measuring the height of the pulse component of the patient's PPG signal by eye. In some embodiments, information about the fluid responsiveness of a patient may be derived through the use of a continuous wavelet transform of the patient's PPG signal. In these embodiments, respiratory components of the PPG signal may be more readily identified and analyzed to derive information related to fluid responsiveness (i.e., because of the correlation between the respiratory components and the fluid responsiveness).

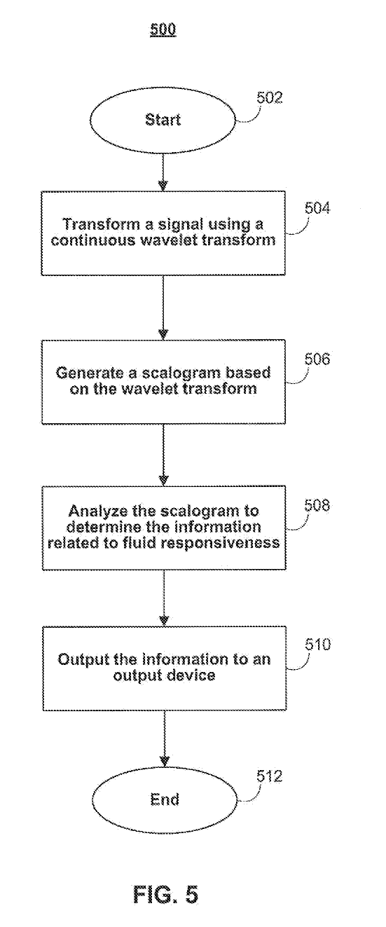

FIG. 5 is a flow chart of illustrative steps involved in determining information related to fluid responsiveness in accordance with some embodiments. Process 500 may begin at step 502 with a signal (e.g., a PPG signal) that may be obtained from sensor 12 that may be coupled to patient 40 (FIG. 2). Alternatively, the PPG signal may be obtained from input signal generator 410, which may include oximeter 420 coupled to sensor 418, which may provide as input signal 416 (FIG. 4) a PPG signal. In an embodiment, the PPG signal may be obtained from patient 40 using sensor 12 or input signal generator 410 in real time. In an embodiment, the PPG signal may have been stored in ROM 52, RAM 54, and/or QSM 72 (FIG. 2) in the past and may be accessed by microprocessor 48 within monitor 14 to be processed.

In an embodiment, at step 504, the received signal may be transformed in any suitable manner. For example, a PPG signal may be transformed using a continuous wavelet transform as described above with respect to, for example, FIG. 3(a)-(d). In an embodiment, at step 506, a scalogram may be generated based at least in part on the transformed signal. For example, processor 412 or microprocessor 48 may perform the calculations associated with the continuous wavelet transform of the PPG signal and the derivation of the scalogram.

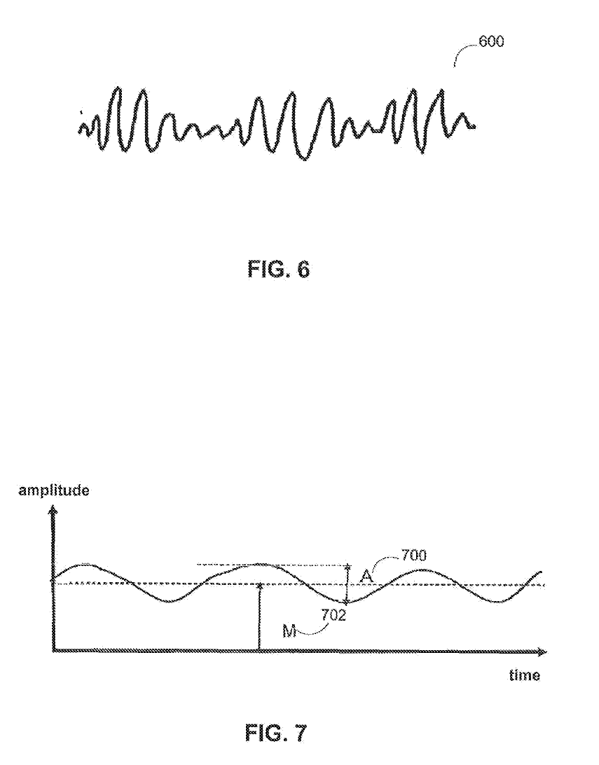

A continuous wavelet transform may be used to obtain characteristic metrics of the respiratory components in a PPG signal, which are, in turn, correlated with fluid responsiveness. In an embodiment at step 508, at least one suitable region, such as a ridge or a band of the scalogram generated in step 506 may be analyzed to determine a level of fluid responsiveness in a patient. For example, the amplitude modulation of a pulse band ridge, such as pulse band ridge 304 in FIG. 3(b), of the scalogram generated in step 506 may be analyzed to determine information related to fluid responsiveness. Changes in the amplitude modulation of the PPG signal correlates with changes in the level of fluid responsiveness. FIG. 6 shows an illustrative amplitude modulation waveform of pulse band ridge 600 in accordance with some embodiments.

The pulse band ridge manifests itself in the scalogram generated from the wavelet transform. The amplitude modulation of the PPG signal may be taken from the amplitude modulation of pulse band ridge 600. By measuring the amplitude variation of pulse band ridge 600, the local pulse modulation of the PPG signal may be extracted. Thus, the level of fluid responsiveness may be captured by the relative pulse amplitude modulation (RPAM), which correlates the amplitude modulation of the PPG signal to the level of fluid responsiveness. Higher values of RPAM may indicate greater levels of fluid responsiveness of a patient. FIG. 7 shows an illustrative pulse band ridge extracted from a scalogram (e.g., the scalogram of FIG. 3(b)) in accordance with some embodiments. The RPAM may be approximated by the properly scaled ratio A/M, where A may be peak-to-peak amplitude modulation 700 of the pulse band ridge and M may be a baseline signal such as mean value 702 of the pulse band ridge. The ratio A/M may be expressed as a percentage.

Other ratios or mathematical expressions may be used to define the RPAM. For example, A may be the standard deviation of the amplitude modulation of pulse band ridge 600, the median absolute value of the amplitude modulation of pulse band ridge 600, any other suitable metric that expresses the amplitude modulation of pulse band ridge 600 to define the RPAM, or any combination thereof. In addition, other characteristic baselines signals or features may be used for M when defining the RPAM. For example, M may be the lower bound of the signal interpolated from the troughs of pulse band ridge 600, the upper bound of the signal interpolated from the peaks of pulse band ridge 600, any other suitable characteristic baseline signal, or any combination thereof.

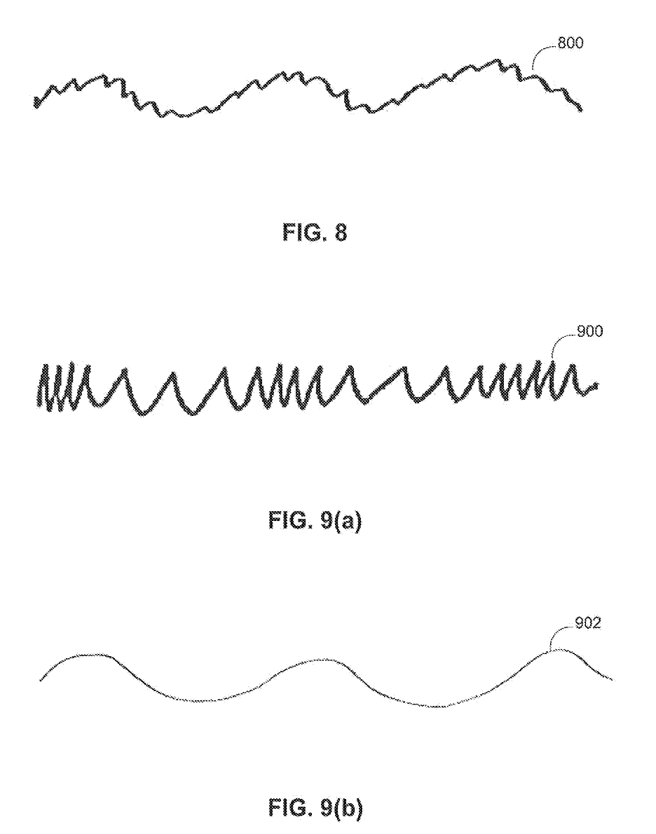

In an embodiment, at step 508, the carrier wave amplitude of the PPG signal may be analyzed to determine information related to fluid responsiveness. Changes in the amplitude of the carrier wave correlates with changes in the level of fluid responsiveness. For example, the carrier wave amplitude may be extracted from the amplitude of the breathing band, such as breathing band 306 in FIG. 3(b), of the scalogram generated in step 506. FIG. 8 shows an illustrative carrier waveform 800 of a PPG signal in accordance with some embodiments. The carrier wave may be indicative of venous return, and the breathing band manifests itself in the scalogram generated from the wavelet transform. The degree of fluid responsiveness may be captured by the relative breathing amplitude modulation (RBAM); in order to extract the respiration modulation component from the breathing band, the component may be divided by a baseline signal to provide a ratio from the breathing band components. The RBAM correlates the amplitude modulation of the carrier wave to the level of fluid responsiveness. Higher values of RBAM may indicate greater levels of fluid responsiveness in a patient. The RBAM may be approximated by the properly scaled ratio B/M, where B may be the amplitude of the breathing band and M may be a baseline signal such as the mean value of the breathing band. The ratio B/M may be expressed as a percentage.

Other ratios or mathematic expressions may be used to define the RBAM. For example, B may be the standard deviation of the amplitude of the breathing band, the median absolute value of the amplitude of the breathing band, any other suitable metric for expressing the amplitude of the breathing band to define the RBAM, or any combination thereof. In addition, other characteristic baselines signals or features may be used for M when defining the RBAM. For example, M may be the lower bound of the signal interpolated from the troughs of the breathing band, the upper bound of the signal interpolated from the peaks of the breathing band, any other suitable characteristic baseline signal, or any combination thereof. In one suitable approach, the RBAM may be expressed by dividing the inverse wavelet transform of the breathing band components by the inverse wavelet transform of the breathing band components below the baseline.

In an embodiment, at step 508, the amplitude of the respiratory sinus arrhythmia (RSA) component of the PPG signal may be analyzed to determine information related to fluid responsiveness. Changes in the amplitude of the RSA component correlates with changes in the level of fluid responsiveness. For example, the RSA component may be derived from the pulse band ridge, such as pulse band ridge 304 in FIG. 3(b), of the scalogram generated in step 506. Further, any band in the transform space indicative of pulse period may provide information for measuring RSA, such as a band at a scale above that of the pulse band, which, though of lower amplitude, may clearly indicate RSA. FIG. 9(a) shows an illustrative signal 900 exhibiting RSA. FIG. 9(b) shows an illustrative RSA waveform derived from a pulse band ridge of a wavelet transform of the signal in FIG. 9(a) in accordance with some embodiments. The pulse band ridge manifests itself in the scalogram generated from the wavelet transform. The amplitude modulation of the RSA may correlate with the amplitude modulation of the pulse band ridge. By measuring the amplitude variation of the pulse band ridge, the local modulation of the RSA waveform may be extracted. The RSA occurs naturally in the variation in the periodicity of the heart beat timing over the respiration cycle. The amplitude modulation of other components of the scalogram generated in step 506 indicative of pulse period may be used to measure RSA in place of or in addition to the amplitude modulation of the pulse band ridge. The level of fluid responsiveness may be captured by the characteristic pulse scale modulation (CPSM), which correlates the amplitude modulation of RSA waveform 900 to the level of fluid responsiveness. Higher values of CPSM may indicate greater levels of fluid responsiveness in a patient. The CPSM may be approximated by the properly scaled ratio C/M, where C may be the amplitude modulation of RSA waveform 900 and M may be a baseline signal such as the mean value of RSA waveform 900. The ratio C/M may be expressed as a percentage.

Other ratios or mathematical expressions may be used to define the CPSM. For example, C may be the standard deviation of the amplitude modulation of RSA waveform 900, the median absolute value of the amplitude modulation of RSA waveform 900, any other suitable metric that expresses the amplitude modulation of RSA waveform 900 to define the CPSM, or any combination thereof. In addition, other characteristic baselines signals or features may be used for M when defining the CPSM. For example, M may be the lower bound of the signal interpolated from the troughs of RSA waveform 900, the upper bound of the signal interpolated from the peaks of RSA waveform 900, any other suitable characteristic baseline signal, or any combination thereof.

It will be understood that any combination of one or more of the RPAM, the RBAM, and/or the CPSM may be used to determine the level of fluid responsiveness. In an embodiment, the information related to fluid responsiveness may be stored in ROM 52, RAM 54, QSM 72, and/or microprocessor 48 within monitor 14 (FIG. 2) and may be accessed by microprocessor 48 to be processed.

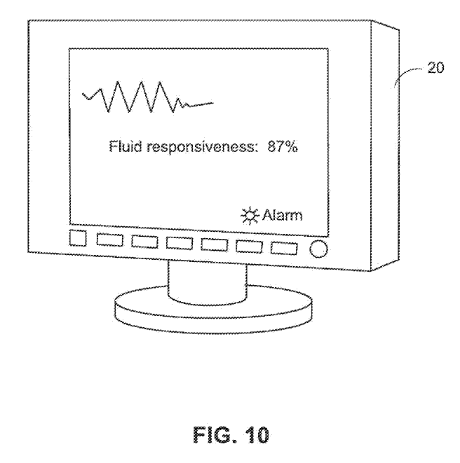

In an embodiment, in step 510, the level of fluid responsiveness determined in step 508 may be outputted to display 20 (FIG. 2), multi-parameter patient monitor 26 (FIG. 1), any other display device communicatively coupled to system 10, or any combination thereof. For example, the level of fluid responsiveness may be displayed on a display such as display 20, as illustrated by FIG. 10. It will be understand that any other metric may be displayed to indicate levels of fluid responsiveness, such as by a status bar, a visual alarm, an audible alarm, any other suitable indication, or any combination thereof. In an embodiment, the level of fluid responsiveness of the patient may be communicated to fluid delivery device 36. Fluid delivery device 36 may accordingly adjust the quantity or concentration of fluid delivered to a patient based at least in part on the level of fluid responsiveness determined above. The level of fluid responsiveness may also be outputted to any other suitable output device, such as a computer, a computer-readable medium, a printer, any other suitable output device, or any combination thereof. Following the output of the degree of fluid responsiveness in step 510, process 500 may advance to step 512 and end.

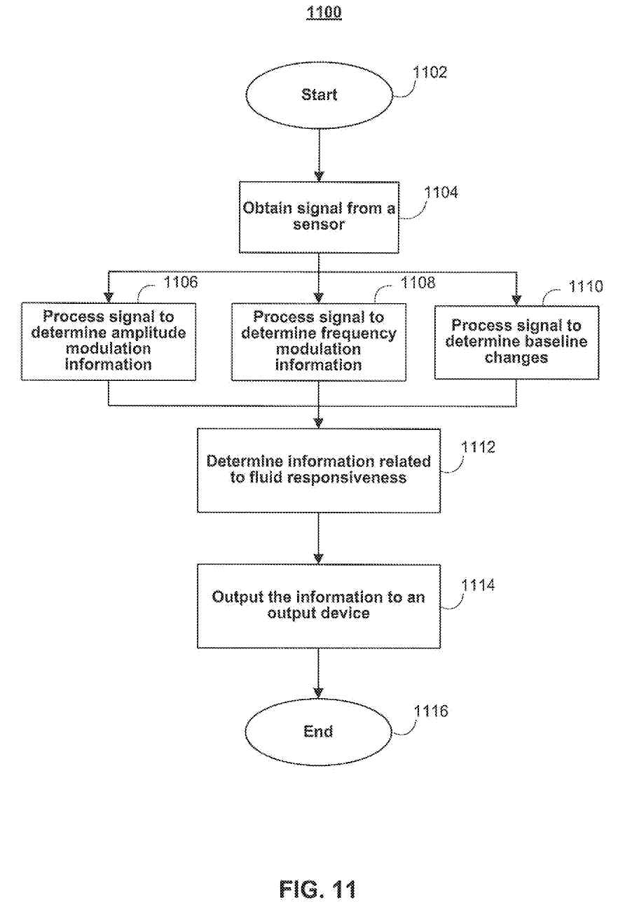

FIG. 11 is a flow chart of illustrative steps involved in determining information related to fluid responsiveness in accordance with some embodiments. Process 1100 may begin at step 1102. At step 1104, a signal (e.g., a PPG signal) may be obtained from sensor 12 that may be coupled to patient 40 (FIG. 2). Alternatively, the PPG signal may be obtained from input signal generator 410, which may include oximeter 420 coupled to sensor 418, which may provide as input signal 416 (FIG. 4) a PPG signal. In an embodiment, the PPG signal may be obtained from patient 40 using sensor 12 or input signal generator 410 in real time. In an embodiment, the PPG signal may have been stored in ROM 52, RAM 54, and/or QSM 72 (FIG. 2) in the past and may be accessed by microprocessor 48 within monitor 14 to be processed.

After receiving the signal at step 1104, the signal may be processed in any suitable manner in order to determine information related to the fluid responsiveness of the patient. In an embodiment, at step 1106, the signal from step 1104 may be processed to determine amplitude modulation information. For example, a PPG signal may be processed by transforming the signal using a continuous wavelet transform as described above with respect to FIG. 3(a)-(d). A continuous wavelet transform may be used to obtain characteristic metrics of the respiratory components in a PPG signal, which are, in turn, correlated with fluid responsiveness. Alternatively, the signal may be processed to determine amplitude modulation information using a discrete wavelet transform, a fast Fourier transform, a Hilbert transform, a discrete cosine transform, any other suitable transform or time-domain signal processing technique, or any combination thereof. Instead of or in addition to the foregoing, the signal may be processed using stochastic or probability-based techniques, such as those based on non-parametric Bayesian estimates, neural networks, any suitable heteroassociative function estimation method, or any combination thereof. Further, the signal may be processed using rule based and adaptive rule based systems such as predicate calculus or propositional, modal, non-monotonic, fuzzy logic, or any combination thereof. In an embodiment, at step 1106, the signal processing may include generating a scalogram based at least in part on the transformed signal. For example, processor 412 or microprocessor 48 may perform the calculations associated with the continuous wavelet transform of the PPG signal and the derivation of the scalogram.

In an embodiment, at step 1108, the signal from step 1104 may be processed to determine frequency modulation information. For example, a PPG signal may be processed by transforming the signal using a continuous wavelet transform as described above with respect to FIG. 3(a)-(d). A continuous wavelet transform may be used to obtain characteristic metrics of the respiratory components in a PPG signal, which are, in turn, correlated with fluid responsiveness. Alternatively, the signal may be processed to determine frequency modulation information using a discrete wavelet transform, a fast Fourier transform, a Hilbert transform, a discrete cosine transform, any other suitable transform, any suitable time domain technique, or any combination thereof. Instead of or in addition to the foregoing, the signal may be processed using stochastic or probability-based techniques, such as those based on non-parametric Bayesian estimates, neural networks, any suitable heteroassociative function estimation method, or any combination thereof. Further, the signal may be processed using rule based and adaptive rule based systems such as predicate calculus or propositional, modal, non-monotonic, fuzzy logic, or any combination thereof. In an embodiment at step 1108, the signal processing may include generating a scalogram based at least in part on the transformed signal. For example, processor 412 or microprocessor 48 may perform the calculations associated with the continuous wavelet transform of the PPG signal and the derivation of the scalogram.

In an embodiment, at step 1110, the signal from step 1104 may be processed to determine baseline changes of the signal. For example, a PPG signal may be processed by transforming the signal using a continuous wavelet transform as described above with respect to FIG. 3(a)-(d). A continuous wavelet transform may be used to obtain characteristic metrics of the respiratory components in a PPG signal, which are, in turn, correlated with fluid responsiveness. Alternatively, the signal may be processed to determine baseline changes of the signal using a discrete wavelet transform, a fast Fourier transform, a Hilbert transform, a discrete cosine transform, any other suitable transform, any suitable time domain technique, or any combination thereof. Instead of or in addition to the foregoing, the signal may be processed using stochastic or probability-based techniques, such as those based on non-parametric Bayesian estimates, neural networks, any suitable heteroassociative function estimation method, or any combination thereof. Further, the signal may be processed using rule based and adaptive rule based systems such as predicate calculus or propositional, modal, non-monotonic, fuzzy logic, or any combination thereof. In an embodiment, at step 1110, the signal processing may include generating a scalogram based at least in part on the transformed signal. For example, processor 412 or microprocessor 48 may perform the calculations associated with the continuous wavelet transform of the PPG signal and the derivation of the scalogram.

In an embodiment, at step 1112, at least one suitable region, such as a ridge or a band of a scalogram that may be generated in steps 1106, 1108, and/or 1110 may be analyzed to determine a level of fluid responsiveness in a patient. Changes in the amplitude modulation, frequency modulation, and/or baseline changes of the pulse band and pulse band ridge correlate with changes in the level of fluid responsiveness. In one embodiment, the amplitude modulation, frequency modulation, and/or baseline changes of either or both the pulse band and the pulse band ridge of the scalogram generated in steps 1106, 1108, and/or 1110 may be analyzed to determine information related to fluid responsiveness. The amplitude modulation, frequency modulation, and/or baseline changes of other components of the PPG signal, such as the dichrotic notch, other suitable waveform reflections, or any combination thereof may also be analyzed to determine changes in fluid responsiveness of a patient. The level of fluid responsiveness may be captured by the RPAM, which correlates the changes in the PPG signal with changes in the degree of fluid responsiveness, thereby providing an indication or measure of fluid responsiveness. Higher values of RPAM may indicate greater levels of fluid responsiveness in a patient. The RPAM may be approximated by the property scaled ratio A/M where A may be the peak-to-peak amplitude modulation of the pulse band ridge and M may be a baseline signal such as the mean value of the pulse band ridge. The ratio A/M may be expressed as a percentage.

Other ratios or mathematical expressions may be used to determine the level of fluid responsiveness. For example, A may be the standard deviation of the amplitude modulation, frequency modulation, and/or baseline changes of the pulse band ridge, the median absolute value of the amplitude modulation, frequency modulation, and/or baseline changes of the pulse band ridge, any other suitable metric that expresses the amplitude modulation, frequency modulation, and/or baseline changes, or any combination thereof. In addition, other characteristic baselines signals or features may be used for M when defining the RPAM. For example, M may be the lower bound of the signal interpolated from the troughs of the pulse band ridge, the upper bound of the signal interpolated from the peaks of the pulse band ridge, any other suitable characteristic baseline signal, or any combination thereof.

In an embodiment, at step 1112, the PPG carrier wave amplitude modulation, frequency modulation, and/or baseline changes may be analyzed to determine information related to fluid responsiveness. Changes in the amplitude modulation, frequency modulation, and/or baseline changes of the carrier wave correlate with changes in the level of fluid responsiveness. The carrier wave correlates with the breathing band, thus the carrier wave information may be extracted from the breathing band of the scalogram that may be generated in steps 1106, 1108, and/or 1110. The degree of fluid responsiveness may be captured by the RBAM, which correlates the changes in the carrier waveform with changes in the degree of fluid responsiveness, thereby providing an indication or measure of fluid responsiveness. Higher values of RBAM may indicate greater levels of fluid responsiveness in a patient. The RBAM may be approximated by the properly scaled ratio B/M, where B may be the amplitude of the breathing band and M may be a baseline signal such as the mean value of the breathing band. The ratio B/M may be expressed as a percentage.

Other ratios or mathematical expressions may be used to determine the level of fluid responsiveness. For example, B may be the standard deviation of the amplitude modulation, frequency modulation, and/or baseline changes of the breathing band, the median absolute value of the amplitude modulation, frequency modulation, and/or baseline changes of the breathing band, any other suitable metric that expresses the amplitude modulation, frequency modulation, and/or baseline changes of the breathing band, or any combination thereof. In addition, other characteristic baselines signals or features may be used for M when defining the RBAM. For example, M may be the lower bound of the signal interpolated from the troughs of the breathing band, the upper bound of the signal interpolated from the peaks of the breathing band, any other suitable characteristic baseline signal, or any combination thereof. Further, the RBAM may also be expressed by dividing the inverse wavelet transform of the breathing band components by the inverse wavelet transform of the breathing band components below the baseline.

In an embodiment, at step 1112, the amplitude modulation, frequency modulation, and/or baseline changes of the RSA component of the PPG signal may be analyzed to determine information related to fluid responsiveness. Changes in the amplitude modulation, frequency modulation, and/or baseline changes of the RSA component correlate with changes in the level of fluid responsiveness. The RSA component manifests itself and may be derived from the pulse band ridge of the scalogram that may be generated in steps 1106, 1108, and/or 1110. Further, any band in the transform space indicative of pulse period may provide information for measuring RSA, such as a band at a scale above that of the pulse band, which, though of lower amplitude, may clearly indicate RSA. The amplitude modulation, frequency modulation, and/or baseline changes of other components of the scalogram generated in steps 1106, 1108, and/or 1110 indicative of pulse period may also be used to measure RSA. The level of fluid responsiveness may be captured by the CPSM, which correlates the changes in the RSA waveform with changes in the degree of fluid responsiveness, thereby providing an indication or measure of fluid responsiveness. Higher values of CPSM may indicate greater levels of fluid responsiveness in a patient. The CPSM may be approximated by the properly scaled ratio C/M, where C may be the modulation of the RSA waveform and M may be a baseline signal such as the mean value of the RSA waveform. The ratio C/M may be expressed as a percentage.

Other ratios or mathematical expressions may be used to define the CPSM. For example, C may be the standard deviation of the amplitude modulation, frequency modulation, and/or baseline changes of the RSA waveform, the median absolute value of the amplitude modulation, frequency modulation, and/or baseline changes of the RSA waveform, any other suitable metric that expresses the amplitude modulation, frequency modulation, and/or baseline changes of the RSA waveform to define the CPSM, or any combination thereof. In addition, other characteristic baselines signals or features may be used for M when defining the CPSM. For example, M may be the lower bound of the signal interpolated from the troughs of the RSA waveform, the upper bound of the signal interpolated from the peaks of the RSA waveform, any other suitable characteristic baseline signal, or any combination thereof.

It will be understood that any combination of one or more of the RPAM, the RBAM, the CPSM, and/or any suitable ratio or calculation may be used to determine the level of fluid responsiveness. In an embodiment, the information related to fluid responsiveness may be stored in ROM 52, RAM 54, QSM 72, and/or microprocessor 48 within monitor 14 (FIG. 2) and may be accessed by microprocessor 48 to be processed.

In an embodiment, in step 1114, the level of fluid responsiveness determined in step 1112 may be outputted to display 20 (FIG. 2), multi-parameter patient monitor 26 (FIG. 1), any other display device communicatively coupled to system 10, or any combination thereof. For example, the level of fluid responsiveness may be displayed on a display such as display 20, as illustrated by FIG. 10. It will be understand that any other metric may be displayed to indicate levels of fluid responsiveness, such as by a status bar, a visual alarm, an audible alarm, any other suitable indication, or any combination thereof. In an embodiment, the level of fluid responsiveness of the patient may be communicated to fluid delivery device 36. Fluid delivery device 36 may accordingly adjust the quantity or concentration of fluid delivered to a patient based at least in part on the level of fluid responsiveness determined above. The level of fluid responsiveness may also be outputted to any other suitable output device, such as a computer, a computer-readable medium, a printer, any other suitable output device, or any combination thereof. Following the output of the degree of fluid responsiveness in step 1114, process 1100 may advance to step 1116 and end.

The foregoing is merely illustrative of the principles of this disclosure and various modifications can be made by those skilled in the art without departing from the scope and spirit of the disclosure.

What is claimed is:

1. A method for measuring fluid responsiveness of a patient, the method comprising:
using processing equipment for:
transforming a physiological signal based at least in part on a wavelet transform to generate a transformed signal,
generating a scalogram from the transformed signal, and
analyzing the scalogram to determine information related to the fluid responsiveness; and
outputting to an output device the information.

2. The method of claim 1, wherein the physiological signal comprises a plethysmographic signal, an electrocardiographic signal, an electromyographic signal, and/or an arterial line trace signal, and/or combinations thereof.

3. The method of claim 1, wherein the analyzing comprises identifying carrier wave information from the physiological signal.

4. The method of claim 1, wherein the analyzing comprises identifying respiratory sinus arrhythmia information from the physiological signal.

5. The method of claim 1, wherein the analyzing comprises identifying amplitude modulation information from the physiological signal.

6. The method of claim 5, wherein the physiological signal is a plethysmographic signal and wherein the analyzing further comprises deriving amplitude modulation information of a pulse band ridge of the scalogram.

7. The method of claim 1, wherein the information comprises a relationship between a characteristic of the amplitude modulation of a particular region of the scalogram to a baseline.

8. The method of claim 7, wherein the characteristic of the amplitude modulation comprises a peak-to-peak amplitude modulation, a standard deviation of the amplitude modulation, and/or a median absolute value of the amplitude modulation, and/or combinations thereof.

9. The method of claim 7, wherein the baseline comprises a mean value of the amplitude modulation, a lower bound of the amplitude modulation, and/or an upper bound of the amplitude modulation, and/or combinations thereof.

10. The method of claim 1, further comprising adjusting a fluid delivery mechanism based at least in part on the information related to fluid responsiveness.

11. A system for measuring fluid responsiveness of a patient, the system comprising:
a fluid delivery mechanism capable of supplying fluid to the patient;
a sensor attached to the patient capable of generating a physiological signal;
a memory;
a processor coupled to the memory capable of:
transforming the physiological signal based at least in part on a wavelet transform stored in the memory to generate a transformed signal,
generating a scalogram from the transformed signal, and
analyzing the scalogram to determine information related to the fluid responsiveness; and
an output device coupled to the processor.

12. The system of claim 11, wherein the physiological signal comprises a plethysmographic signal, an electrocardiographic signal, an electromyographic signal, and/or an arterial line trace signal, and/or combinations thereof.

13. The system of claim 11, wherein the processor is further capable of identifying carrier wave information of the physiological signal.

14. The system of claim 11, wherein the analyzing comprises identifying respiratory sinus arrhythmia information from the physiological signal.

15. The system of claim 11, wherein the analyzing comprises identifying amplitude modulation information of the physiological signal.

16. The system of claim 15, wherein the physiological signal is a plethysmographic signal and wherein the amplitude modulation information comprises amplitude modulation information of a pulse band ridge of the scalogram.

17. The system of claim 11, wherein the information comprises a relationship between a characteristic of the amplitude modulation of a particular region of the scalogram to a baseline.

18. The system of claim 11, wherein the processor is coupled to the fluid delivery system and wherein the processor is further capable of adjusting the fluid delivery mechanism based at least in part on the information related to fluid responsiveness.

19. A method for measuring fluid responsiveness of a patient, the method comprising:
obtaining using a sensor attached to the patient a physiological signal;
processing using a processor the physiological signal to determine amplitude modulation information of the physiological signal;
processing using the processor the physiological signal to determine frequency modulation information of the physiological signal;
determining using the processor information related to the fluid responsiveness; and
outputting the information to an output device.

20. The method of claim 19, wherein the physiological signal comprises a plethysmographic signal, an electrocardiographic signal, an electromyographic signal, and/or an arterial line trace signal, and/or combinations thereof.
